(19) United States
(12) Patent Application Publication
(10) Pub. No.: US 2019/0209822 A1
(43) Pub. Date: Jul. 11, 2019

(54) MICROSTRUCTURE USING WATERPROOF THIN FILM AND MANUFACTURING METHOD THEREFOR

(71) Applicant: JUVIC INC., Seoul (KR)

(72) Inventors: Hyung IL JUNG, Seoul (KR); Chengguo LI, Seoul (KR); Yonghao MA, Seoul (KR)

(21) Appl. No.: 16/300,531

(22) PCT Filed: May 10, 2017
(86) PCT No.: PCT/KR2017/004812

§ 371 Date: Nov. 9, 2018

(30) Foreign Application Priority Data
May 9, 2016 (KR) 10-2016-0056490

(51) Int. Cl. A61M 37/00; C25D 5/56; C25D 3/02

(52) U.S. Cl. CPC A61M 37/0015; C25D 5/56; A61M 2037/0053; A61M 2037/0023; C25D 3/02

(57) ABSTRACT

The present invention relates to a microstructure and a manufacturing method therefor, the method comprising the steps of: (a) forming a water-soluble microstructure core on a substrate; and (b) forming a waterproof thin film on the water-soluble microstructure core.

### TECHNICAL FIELD

The present invention relates to a microstructure using a waterproof thin film and a manufacturing method therefor.

### BACKGROUND ART

Since a general hypodermic injection needle causes many people to develop aichmophobia and induces pain at the time of use or leaves trauma, hypodermic injection needle has become a limiting factor in such uses as clinical blood collection or diagnosis and drug injection in a clinical setting. In order to solve this problem, microneedles have emerged as an alternative.

Microneedles may be manufactured by using a mold, and may be manufactured by molding a viscous composition. Further, examples of microneedles include a solid type, a lancet type, and a hollow type, and in order to manufacture a lancet type or hollow type among the microneedles in the related art, there is a limitation in which a non-water-soluble polymer (such as a SU-8 photoresist) needs to be used before a process of depositing a metal. In this case, when microneedles are manufactured by using an expensive SU-8 photoresist, and the like, there are limitations in that the manufacturing process is complicated and precise temperature adjustment is needed.

### DISCLOSURE

### Technical Problem

The present invention has been made in an effort to provide a method for manufacturing a microstructure, the method comprising the steps of: (a) forming a water-soluble microstructure core on a substrate; and (b) forming a waterproof thin film on the water-soluble microstructure core.

However, a technical problem to be achieved by the present invention is not limited to the aforementioned problem, and other problems that are not mentioned may be clearly understood by a person skilled in the art from the following description.

### Technical Solution

The present invention provides a method for manufacturing a microstructure, the method comprising the steps of: (a) forming a water-soluble microstructure core on a substrate; and (b) forming a waterproof thin film on the water-soluble microstructure core.

The water-soluble microstructure core may include a drug or an additive.

The waterproof thin film may include one or more waterproof polymers selected from the group consisting of a parylene-based polymer, an ethylene-based polymer, an ester-based polymer, an acrylic polymer, an acetyl-based polymer, a styrene-based polymer, a Teflon-based polymer, a vinyl chloride-based polymer, a urethane-based polymer, a nylon-based polymer, a sulfone-based polymer, an epoxy-based polymer, a fluorine-based polymer, and a silicone-based polymer.

The waterproof thin film may have a thickness of 10 nm to 10 μm.

The method may further include the step of (c) depositing a metal onto the waterproof thin film, and then plating the waterproof thin film.

The method may further include the step of (d) separating the substrate, and then removing the water-soluble microstructure core.

An embodiment of the present invention includes a microstructure including: a substrate; a water-soluble microstructure core formed on the substrate; and a waterproof thin film formed on the water-soluble microstructure core.

The water-soluble microstructure core may include a drug or an additive.

The waterproof thin film may include one or more waterproof polymers selected from the group consisting of a parylene-based polymer, an ethylene-based polymer, an ester-based polymer, an acrylic polymer, an acetyl-based polymer, a styrene-based polymer, a Teflon-based polymer, a vinyl chloride-based polymer, a urethane-based polymer, a nylon-based polymer, a sulfone-based polymer, an epoxy-based polymer, a fluorine-based polymer, and a silicone-based polymer.

The waterproof thin film may have a thickness of 10 nm to 10 μm.

The microstructure may further include a metal thin film plated on the waterproof thin film.

Another embodiment of the present invention provides a microstructure including: a hollow-type waterproof thin film; and a metal thin film plated on the hollow-type waterproof thin film.

The waterproof thin film may include one or more waterproof polymers selected from the group consisting of a parylene-based polymer, an ethylene-based polymer, an ester-based polymer, an acrylic polymer, an acetyl-based polymer, a styrene-based polymer, a Teflon-based polymer, a vinyl chloride-based polymer, a urethane-based polymer, a nylon-based polymer, a sulfone-based polymer, an epoxy-based polymer, a fluorine-based polymer, and a silicone-based polymer.

The waterproof thin film may have a thickness of 10 nm to 10 μm.

### Advantageous Effects

The present invention relates to a microstructure using a waterproof thin film and a manufacturing method therefor, and has advantages in that when a solid-type microstructure is manufactured, a drug or an additive included in a water-soluble microstructure core can be effectively delivered to the skin or cells due to a waterproof thin film, and when a lancet-type or hollow-type microstructure is manufactured, a metal is easily deposited due to the waterproof thin film even though a water-soluble microstructure core is used. Further, the present invention has an advantage in that when a hollow-type microstructure is manufactured, a water-soluble microstructure is easily removed.

Accordingly, a microstructure using the waterproof thin film according to the present invention may be used variously for not only a use of delivering a drug or an additive to skin or cells, but also a use of sampling a body fluid, and the like.

### DESCRIPTION OF DRAWINGS

FIG. 1 is a flowchart illustrating a method for manufacturing a microstructure according to various embodiments.

FIG. 2 is a view illustrating a method for manufacturing a solid-type microstructure according to an embodiment of the present invention.

FIG. 3 is a view illustrating a process of delivering a solid-type microstructure manufactured according to an embodiment of the present invention to a target cell or the skin.

FIG. 4 is a view illustrating a method for manufacturing a lancet-type microstructure according to an embodiment of the present invention.

FIG. 5 is a view illustrating a method for manufacturing a hollow-type microstructure according to an embodiment of the present invention.

FIG. 6 is (a) an SEM photograph of a solid-type microstructure manufactured in Example 1 and (b) an SEM photograph illustrating a result in which the waterproof performance of the solid-type microstructure manufactured in Example 1 is evaluated.

FIG. 7 is (a) an SEM photograph of a lancet-type microstructure manufactured in Example 2 and (b) an SEM photograph of a hollow-type microstructure manufactured in Example 3.

### MODES OF THE INVENTION

The inventors of the present invention manufactured a microstructure which is characterized by coating a waterproof thin film onto a water-soluble microstructure core without using a non-water-soluble polymer as in the related art, thereby completing the present invention.

Hereinafter, examples of the present invention will be described in detail with reference to the accompanying drawings so that a person with ordinary skill in the art to which the present invention pertains can easily carry out the present invention. The present invention can be realized in various forms, and is not limited to the examples described herein.

In the drawings, in order to clearly express several layers and regions, their thicknesses are enlarged. Moreover, in the drawings, the thicknesses of some layers and regions are exaggerated for convenience of explanation.

Hereinafter, formation of any configuration on the top (or bottom) of a substrate means not only that the configuration is formed in contact with an upper surface (or a lower surface) of the substrate, but may also mean that there are other configurations between the substrate and the configuration formed on the top (or bottom) of the substrate.

Hereinafter, the present invention will be described in detail.

Method for Manufacturing Microstructure

The present invention provides a method for manufacturing a microstructure, the method comprising the steps of: (a) forming a water-soluble microstructure core on a substrate; and (b) forming a waterproof thin film on the water-soluble microstructure core. In this case, the manufactured microstructure is a solid-type microstructure.

Optionally, the method may further include the step of (c) depositing a metal onto the waterproof thin film, and then plating the waterproof thin film. In this case, the manufactured microstructure is a lancet-type microstructure.

Thereafter, optionally, the method may further include the step of (d) separating the substrate, and then removing the water-soluble microstructure core. In this case, the manufactured microstructure is a hollow-type microstructure.

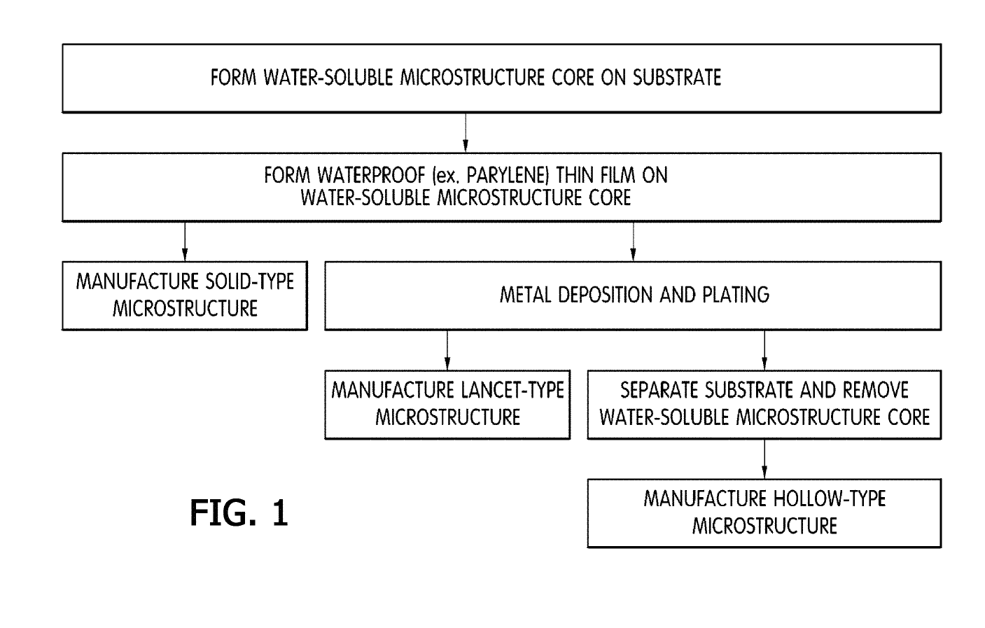

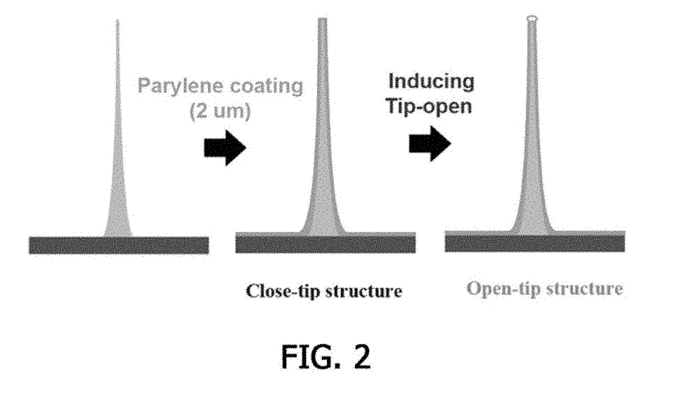

FIGS. 1 and 2 illustrate a method for manufacturing a microstructure according to an embodiment of the present invention.

As illustrated in FIG. 1, the method for manufacturing a solid-type microstructure according to an embodiment of the present invention comprises the steps of: forming a water-soluble microstructure core on a substrate; and forming a waterproof thin film on the water-soluble microstructure core.

Specifically, as illustrated in FIG. 2, the solid-type microstructure according to an embodiment of the present invention may be classified into a form having a closed upper end portion (tip) or an open upper end portion (tip). Specifically, when a waterproof thin film is entirely formed on the water-soluble microstructure core, a solid-type microstructure having a closed upper end portion (tip) can be manufactured, and when a portion of the water-soluble microstructure core is exposed by cutting an upper end portion of an end portion after forming a waterproof thin film entirely on the water-soluble microstructure core, a solid-type microstructure having an open upper end portion (tip) can be manufactured.

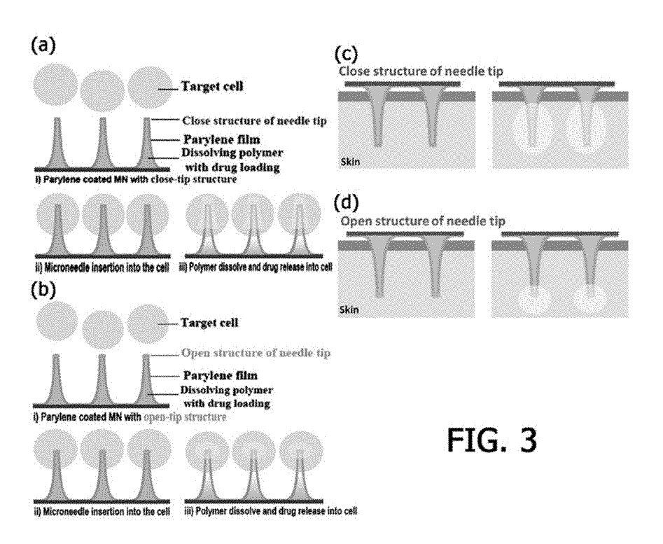

FIG. 3 is a view illustrating a process of delivering a solid-type microstructure manufactured according to an embodiment of the present invention to a target cell or the skin.

As illustrated in FIG. 3(A) or 3(C), when a solid-type microstructure having a closed upper end portion (tip) is allowed to penetrate into a target cell or the skin, only in the case where the solid-type microstructure becomes a porous structure due to the deterioration in waterproof performance of the waterproof thin film, can a drug or an additive included in the water-soluble microstructure core between the porous structures of the waterproof thin film be released and effectively delivered to a target cell or the skin.

That is, it is preferred that a certain degree of waterproof performance of the solid-type microstructure having a closed upper end portion (tip) be maintained by adjusting the thickness, material, and the like of the waterproof thin film.

As illustrated in FIG. 3(B) or 3(D), when the solid-type microstructure having an open upper end portion (tip) is allowed to penetrate into a target cell or the skin, a drug or an additive in the water-soluble microstructure core can be released through a cut upper end portion and effectively transferred to the target cell or the skin.

That is, a solid-type microstructure having an open upper end portion (tip) can effectively transfer a drug or an additive included in a water-soluble microstructure core to a target cell or the skin even if the thickness, material, and the like of a water-soluble thin film are not adjusted, unlike a solid-type microstructure having a closed upper end portion (tip).

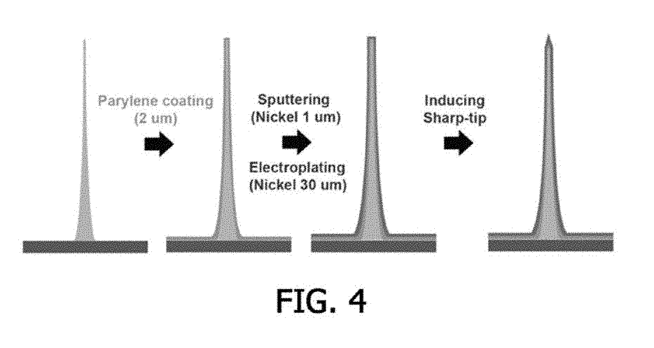

FIGS. 1 and 4 illustrate a method for manufacturing a solid-type microstructure according to an embodiment of the present invention.

As illustrated in FIG. 1, the method for manufacturing a lancet-type microstructure according to an embodiment of the present invention further comprises the step of depositing a metal onto the waterproof thin film, and then plating the waterproof thin film after manufacturing a solid-type microstructure, as described above. Thereafter, as illustrated in FIG. 4, the method may further comprise the step of sharply molding an upper end portion (tip) of an end portion in the lancet-type microstructure according to an embodiment of the present invention.

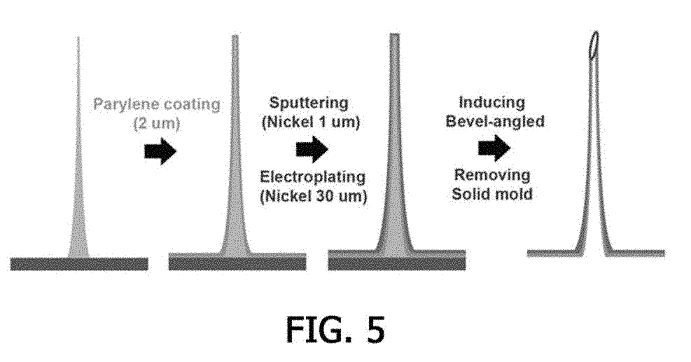

FIGS. 1 and 5 illustrate a method for manufacturing a hollow-type microstructure according to an embodiment of the present invention.

As illustrated in FIG. 1, the method for manufacturing a hollow-type microstructure according to an embodiment of the present invention further comprises the step of separating the substrate, and then removing the water-soluble microstructure core after manufacturing a lancet-type microstructure, as described above. Thereafter, as illustrated in FIG. 5, the method may further comprise the step of obliquely cutting an upper end portion (tip) of an end portion in the hollow-type microstructure according to an embodiment of the present invention.

First, the method for manufacturing a microstructure according to an embodiment of the present invention comprises the step of forming a water-soluble microstructure core on a substrate [Step (a)].

Even though a water-soluble microstructure core is used, the present invention has an advantage in that the drug or the additive included in the water-soluble microstructure core can be effectively delivered to the skin or the cell, and a metal is easily deposited, due to a waterproof thin film.

The substrate is used for a use of supporting the water-soluble microstructure core.

The substrate may have various surface shapes. Further, the substrate may support the water-soluble microstructure core immediately on the substrate without forming a pillar, and when a microstructure is delivered to the skin or a cell by forming one pillar having any of various shapes such as a cylinder, a truncated cone, a cone, or a semi-circle to support the water-soluble microstructure core, the degree can be adjusted.

The water-soluble microstructure core may be formed by various publicly known methods, may be formed by using a mold, and may be formed by molding a viscous composition. In this case, the molding may also be performed by any of various publicly known methods such as molding, drawing, blowing, suctioning, application of centrifugal force, or application of a magnetic field.

The water-soluble microstructure core has a property of being dissolved in water, and may include a biocompatible or biodegradable material.

The term “biocompatible material” used herein refers to a material that is substantially non-toxic to the human body, is chemically inert, and is non-immunogenic, and the term “biodegradable material” used herein refers to a material which can be degraded by body fluids, microorganisms, or the like in vivo.

Specifically, as the biocompatible or biodegradable material, it is possible to use hyaluronic acid, polyester, polyhydroxyalkanoates (PHAs), poly(α-hydroxy acid), poly(β-hydroxy acid), poly(3-hydroxybutyrate-co-valerate) (PHBV), poly(3-hydroxyproprionate) (PHP), poly(3-hydroxyhexanoate) (PHH), poly(4-hydroxy acid), poly(4-hydroxybutyrate), poly(4-hydroxyvalerate), poly(4-hydroxyhexanoate), poly(ester amide), polycaprolactone, polylactide, polyglycolide, poly(lactide-co-glycolide) (PLGA), polydioxanone, polyorthoester, polyether ester, polyanhydride, poly(glycolic acid-co-trimethylene carbonate), polyphosphoester, polyphosphoester urethane, poly(amino acid), polycyanoacrylate, poly(trimethylene carbonate), poly(iminocarbonate), poly(tyrosine carbonate), polycarbonate, poly(tyrosine arylate), polyalkylene oxalates, polyphosphazenes, PHA-PEG, an ethylene vinyl alcohol copolymer (EVOH), polyurethane, silicone, polyester, polyolefin, a polyisobutylene and ethylene-alphaolefin copolymer, a styrene-isobutylene-styrene triblock copolymer, an acrylic polymer or copolymer, a vinyl halide polymer or copolymer, polyvinyl chloride, polyvinyl pyrrolidone, polyvinyl ether, polyvinyl methyl ether, polyvinylidene halides, polyvinylidene fluoride, polyvinylidene chloride, polyfluoroalkenes, polyperfluoroalkenes, polyacrylonitrile, polyvinyl ketone, polyvinyl aromatics, polystyrene, polyvinyl ester, polyvinyl acetate, an ethylene-methyl methacrylate copolymer, an acrylonitrile-styrene copolymer, an ABS resin and ethylene-vinyl acetate copolymer, polyamide, an alkyd resin, polyoxymethylene, polyimide, polyether, polyacrylate, polymethacrylate, polyacrylic acid-co-maleic acid, chitosan, dextran, cellulose, carboxymethyl cellulose, heparin, alginate, inulin, starch or glycogen.

Since the water-soluble microstructure core may include a drug or an additive, the drug or the additive can be effectively delivered to the skin or a cell.

As the drug, a publicly known drug can be used, and for example, the drug may be a chemical, a protein medicine, a peptide medicine, nucleic acid molecules for gene therapy, nanoparticles, or the like. For example, the drug may be an anti-inflammatory agent, an analgesic agent, an antiarthritic agent, an antispasmodic agent, an antidepressant, an antipsychotic drug, a tranquilizer, an antianxiety drug, a narcotic antagonist, an anti-Parkinson's disease drug, a cholinergic agonist, an anticancer drug, an anti-angiogenesis inhibitor, an immunosuppressant, an antiviral agent, an antibiotic, an appetite suppressant, an analgesic agent, an anticholinergic drug, an antihistaminic agent, an anti-migraine agent, a hormone drug, a coronary, cerebrovascular or peripheral vasodilator, a contraceptive, an antithrombotic drug, a diuretic drug, an antihypertensive drug, a cardioprotective agent, a cosmetic ingredient (for example, an anti-wrinkle agent, an anti-skin-aging agent, or a skin whitening agent), or the like, but the present invention is not limited thereto.

Since the water-soluble microstructure core may be implemented under a non-heating treatment condition, the manufacture of the water-soluble microstructure core can be applied even if the drug is a drug vulnerable to heat, such as a protein medicine, a peptide medicine, nucleic acid molecules for gene therapy, or a vitamin (preferably, vitamin C).

The protein/peptide medicine may be a hormone, a hormone analogue, an enzyme, an enzyme inhibitor, a signaling protein or fragments thereof, an antibody or fragments thereof, a single-chain antibody, a binding protein or binding domains thereof, an antigen, an adhesion protein, a structural protein, a regulatory protein, a toxoprotein, a cytokine, a transcriptional regulatory factor, a blood coagulation factor, a vaccine, or the like, but the present invention is not limited thereto. More specifically, the protein/peptide medicine may be insulin, an insulin-like growth factor 1 (IGF-1), a growth hormone, erythropoietin, granulocyte-colony stimulating factors (G-CSFs), granulocyte/macrophage-colony stimulating factors (GM-CSFs), interferon-alpha, interferon-beta, interferon-gamma, interleukin-1 alpha and beta, interleukin-3, interleukin-4, interleukin-6, interleukin-2, epidermal growth factors (EGFs), calcitonin, an adrenocorticotropic hormone (ACTH), a tumor necrosis factor (TNF), atobisban, buserelin, cetrorelix, deslorelin, desmopres sin, dynorphin A (1-13), elcatonin, eleidosin, eptifibatide, growth hormone releasing hormone-II (GHRH-II), gonadorelin, goserelin, histrelin, leuprorelin, lypressin, octreotide, oxytocin, pitressin, secretin, sincalide, terlipressin, thymopentin, thymosine α1, triptorelin, bivalirudin, carbetocin, cyclosporine, exedine, lanreotide, a luteinizing hormone-releasing hormone (LHRH), nafarelin, a parathyroid hormone, pramlintide, T-20 (enfuvirtide), thymalfasin, or ziconotide.

The additive is usually one of various materials for enhancing the effect or stability of the drug, and it is possible to use a publicly known immunity-inducing agent for increasing the efficacy of the drug or sugars such as trehalose for enhancing the stability of the drug. In addition, energy may also be used. In this case, the water-soluble microstructure core may be used for transferring or delivering an energy form such as heat energy, light energy, or electric energy.

For example, in photodynamic therapy, the water-soluble microstructure core may be used to induce light to specific locations within the body, such that the light can act directly on a tissue, or on an intermediary, such as light-sensitive molecules in the photodynamic therapy.

Next, the method for manufacturing a microstructure according to an embodiment of the present invention comprises the step of forming a waterproof thin film on the water-soluble microstructure core [Step (b)].

The method has advantages in that when a solid-type microstructure is manufactured, a drug or an additive included in a water-soluble microstructure core can be effectively delivered to the skin or cells due to a waterproof thin film, and when a lancet-type or hollow-type microstructure is manufactured, a metal is easily deposited due to a waterproof thin film even though a water-soluble microstructure core is used.

Particularly, when a solid-type microstructure having a closed upper end portion (tip) is manufactured by using the waterproof thin film and is allowed to penetrate into a target cell or the skin, only in the case where the solid-type microstructure becomes a porous structure due to the deterioration in waterproof performance of the waterproof thin film, can a drug or an additive included in the water-soluble microstructure core between the porous structures of the waterproof thin film be released and effectively delivered to a target cell or the skin. Accordingly, the thickness, material, and the like of the waterproof thin film need to be adjusted such that the waterproof thin film maintains a certain degree of waterproof performance.

The waterproof thin film preferably includes one or more waterproof polymers selected from the group consisting of a parylene-based polymer, an ethylene-based polymer, an ester-based polymer, an acrylic polymer, an acetyl-based polymer, a styrene-based polymer, a Teflon-based polymer, a vinyl chloride-based polymer, a urethane-based polymer, a nylon-based polymer, a sulfone-based polymer, an epoxy-based polymer, a fluorine-based polymer, and a silicone-based polymer, and more preferably a parylene-based polymer, but is not limited thereto.

Specifically, the parylene-based polymer may be one or more selected from the group consisting of poly(para-xylene), poly(2-chloro-1,4-dimethyl benzene), poly(1,4-dichloro-2,5-dimethylbenzene), and poly(2-fluoro-1,4-dimethylbenzene), and is preferably parylene or poly(para-xylene) represented by the following Chemical Formula 1, but is not limited thereto.

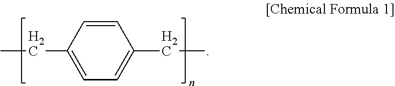

The waterproof thin film formed of parylene is coated in a thin film state onto the surface of a substrate by evaporating, thermally decomposing, and polymerizing parylene in a powder state. The waterproof thin film formed of parylene has biocompatibility, heat resistance, and corrosion resistance in addition to the waterproof performance, and can form a thin film having a uniform thickness can be formed regardless of a shape of the surface of the substrate without generating defects such as empty holes. Accordingly, the waterproof thin film formed of parylene is generally useful in the fields of medicine and biology. Recently, the parylene material has been approved as a biocompatible material by the FDA.

Meanwhile, when a solid-type microstructure is manufactured by using a biodegradable polymer while having waterproof performance as the waterproof thin film, the waterproof thin film has an advantage in that after the waterproof function is performed for a certain period of time according to the specific material, the waterproof thin film may be degraded within the skin or the cell along with a drug or an additive included in the water-soluble microstructure core.

Accordingly, the solid-type microstructure manufactured as described above may be applied onto a mask pack sheet, and as an embodiment, when a solid-type microstructure is packed by including a mask pack sheet and a beauty liquid together while the solid-type microstructure is applied onto the mask pack sheet, the waterproof thin film of the solid-type microstructure needs to perform a waterproof function at least until the mask pack sheet is opened while the waterproof thin film of the solid-type microstructure is not degraded in advance, and therefore it is preferred to use a material having a waterproof performance that lasts for a long period of time. As another embodiment, when a solid-type microstructure is applied to a mask pack sheet including a beauty liquid after the solid-type microstructure is packed by including the mask pack sheet and the beauty liquid together and the packing of the mask pack sheet is opened while the solid-type microstructure is not applied onto the mask pack sheet, it is sufficient for the waterproof thin film to perform the waterproof function for the application time and the skin adhesion time, and therefore a material whose waterproof performance lasts for a short period of time may be used. As still another embodiment, when a beauty liquid is mixed with a mask pack sheet to which a solid-type microstructure is applied after the mask pack sheet is packed and the beauty liquid is separately packed while the solid-type microstructure is applied onto the mask pack sheet, it is sufficient for the waterproof thin film to perform the waterproof function for the mixing time and the skin adhesion time, and therefore a material whose waterproof performance lasts for a short period of time may be used.

A thickness of the waterproof thin film is preferably 10 nm to 10 μm, and more preferably 10 nm to 1 μm, but is not limited thereto. When a solid-type microstructure having a closed upper end portion (tip) is manufactured by using the waterproof thin film, a porous structure cannot be formed in a target cell or the skin if the thickness of the waterproof thin film is too large, and therefore the thickness is preferably 10 nm to 100 nm, but is not limited thereto.

The microstructure manufactured in Steps (a) and (b) is a solid-type microstructure, specifically, a solid-type microstructure having a closed upper end portion (tip). In the solid-type microstructure having the closed upper end portion (tip), a solid-type microstructure having an open upper end portion (tip) can be manufactured when a portion of the water-soluble microstructure core is exposed by cutting the upper end portion (tip) of the end portion.

Optionally, the method for manufacturing a microstructure according to an embodiment of the present invention may further comprise the step of depositing a metal onto the waterproof thin film, and then plating the waterproof thin film [Step (c)].

As the metal for plating, any metal known in the art may be used as long as the metal is a metal that has no toxicity or carcinogenicity, is not rejected by the human body, has good mechanical properties such as tensile strength, elastic modulus, and wear resistance, and is a bioapplicable metal with corrosion resistance capable of withstanding a corrosive environment in the human body. Specifically, the metal for plating is preferably one or more selected from the group consisting of stainless steel, aluminum, chromium, nickel, gold, silver, copper, titanium, cobalt, and alloys thereof, but is not limited thereto.

Before the plating, the step of depositing a seed layer for electrical activation may be further added.

Various forms and mechanical properties of a hollow-type microneedle to be finally manufactured can be adjusted by adjusting the plating thickness.

The microstructure manufactured in Steps (a) to (c) is a lancet-type microstructure. The method may further comprise the step of sharply molding the upper end portion (tip) of the end portion in the lancet-type microstructure.

Optionally, the method for manufacturing a microstructure according to an embodiment of the present invention may further comprise the step of separating the substrate, and then removing the water-soluble microstructure core [Step (d)].

For the removal of the water-soluble microstructure core, the water-soluble microstructure core can be dissolved by using an organic solvent, combusted, or physically removed, after the substrate is separated. In this case, since the water-soluble microstructure core is formed of a biocompatible or biodegradable material having a property of being dissolved in water without using a non-water-soluble polymer, the water-soluble microstructure core is easily removed.

The microstructure manufactured in Steps (a) to (d) is a hollow-type microstructure. The method may further include the step of obliquely cutting an upper end portion (tip) of an end portion in the hollow-type microstructure.

Microstructure

The present invention provides a microstructure including: a substrate; a water-soluble microstructure core formed on the substrate; and a waterproof thin film formed on the water-soluble microstructure core. The microstructure is a solid-type microstructure.

Further, the present invention provides a microstructure including: a substrate; a water-soluble microstructure core formed on the substrate; a waterproof thin film formed on the water-soluble microstructure core; and a metal thin film plated on the waterproof thin film. The microstructure is a lancet-type microstructure. In this case, in the lancet-type microstructure, the upper end portion (tip) of the end portion may be in a sharply molded form.

In addition, the present invention provides a microstructure including: a hollow-type waterproof thin film; and a metal thin film plated on the hollow-type waterproof thin film. The microstructure is a hollow-type microstructure. In this case, in the hollow-type microstructure, the upper end portion (tip) of the end portion may be in an obliquely cut form.

Specific details of the configuration constituting the microstructure are the same as those described above in the method for manufacturing a microstructure.

The microstructure can be used as a microblade, a microknife, a microfiber, a microspike, a microprobe, a microbarb, a microarray, a microelectrode, or the like, in addition to a microneedle.

The microstructure may have various forms such as various effective lengths and upper end portion and lower end portion diameters of the end portion.

The term “effective length” as used herein refers to a vertical length from an upper end portion of the end portion to the surface of a substrate, and may be a length of 100 to 10,000 μm, 200 to 10,000 μm, 300 to 8,000 μm, or 500 to 2,000 μm.

Furthermore, the term “upper end portion (tip) of the end portion” used herein refers to an end of an end portion having a minimum diameter, which may be a diameter of 1 to 500 μm, 2 to 300 μm, or 5 to 100 μm. Further, the term “lower end portion of the end portion” used herein refers to an end of an end portion having a maximum diameter, which may be 50 to 1,000 μm.

As described above, the present invention relates to a microstructure using a waterproof thin film and a manufacturing method therefor, and has advantages in that when a solid-type microstructure is manufactured, a drug or an additive included in a water-soluble microstructure core can be effectively delivered to the skin or cells due to a waterproof thin film, and when a lancet-type or hollow-type microstructure is manufactured, a metal is easily deposited due to a waterproof thin film even though a water-soluble microstructure core is used. Further, the present invention has an advantage in that when a hollow-type microstructure is manufactured, a water-soluble microstructure is easily removed.

Accordingly, a microstructure using the waterproof thin film according to the present invention may be used variously for not only a use of delivering a drug or an additive to the skin or cells, but also a use of sampling a body fluid, and the like.

Hereinafter, preferred Examples for helping with understanding of the present invention will be suggested. However, the following Examples are provided only so that the present invention may be more easily understood, and the content of the present invention is not limited by the following Examples.

### EXAMPLES

### Example 1

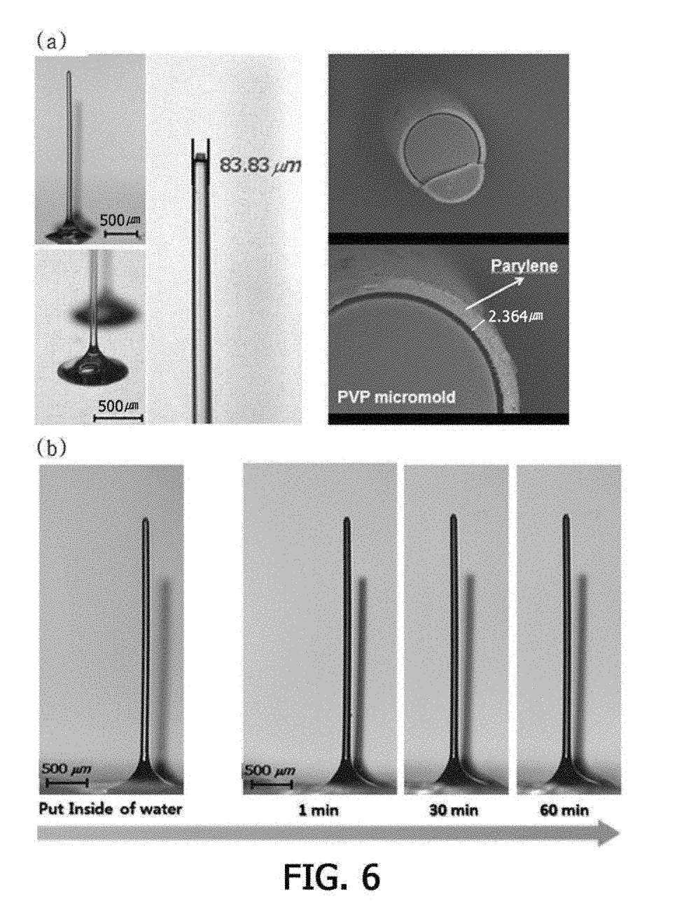

A viscous composition of polyvinylpyrrolidone (PVP, 36 kDa, Sigma, USA) was prepared by discharging the polyvinylpyrrolidone at 0.9 kgfcm⁻¹ for 5 minutes through a dispenser (ML-5000X, Musashi, Japan) on a lower substrate formed of glass. Thereafter, after an upper substrate formed of glass was brought into contact with the viscous composition, the upper substrate was drawn at a speed of 5 mm/min, and the viscous composition was cured by applying symmetric air blowing to the upper substrate at room temperature for 5 minutes. Thereafter, a water-soluble microstructure core formed of polyvinylpyrrolidone (PVP, 36 kDa, Sigma, USA) was manufactured by separating the substrates at a speed of 30 mm/min. Thereafter, a solid-type microstructure was manufactured by entirely depositing a waterproof thin film formed of parylene, which had a thickness of about 2 μm (effective length=about 4 mm, diameter of the upper end portion (tip) of the end portion=about 83 μm, and diameter of the lower end portion of the end portion=about 300 to 500 μm) (see FIG. 6(A)).

As a result of evaluating the waterproof performance for 60 minutes by putting the solid-type microstructure manufactured in Example 1 into water, it could be confirmed that the manufactured solid-type microstructure maintained a stable form in water for 60 minutes while a water-soluble microstructure core formed of polyvinylpyrrolidone (PVP) was not dissolved in water due to a waterproof thin film formed of parylene (see FIG. 6(B)).

### Example 2

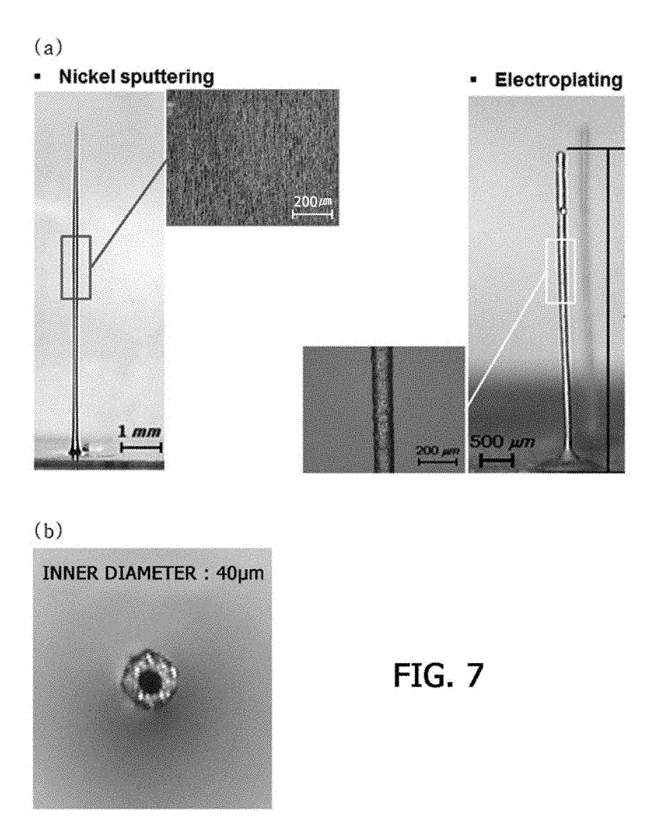

After a silver seed layer for electrical activation was deposited onto the solid-type microstructure manufactured in Example 1, nickel was deposited onto the solid-type microstructure in a nickel bath at 52° C. at a constant current density of 2.8 mA/cm² for 150 minutes, and then the solid-type microstructure was plated. Thereafter, a lancet-type microstructure was manufactured by sharply molding the upper end portion (tip) of the upper portion using a laser cutting machine (K2 Laser System, Korea) (see FIG. 7(A)).

### Example 3

After the lancet-type microstructure manufactured in Example 2 was separated from the substrate, the upper end portion (tip) of the end portion was cut at an angle of 15° obliquely with respect to the vertical line by using a laser cutting machine (K2 Laser System, Korea), and then the water-soluble microstructure core was removed by immersing the lancet-type microstructure in water to dissolve all of the water-soluble microstructure core, thereby manufacturing a hollow-type microstructure (see FIG. 7(B)).

The above description of the present invention is provided for illustrative purposes, and those skilled in the art to which the present invention pertains will understand that the present invention can be easily modified into other specific forms without changing the technical spirit or essential features of the present invention. Therefore, it should be understood that the above-described Examples are illustrative only in all aspects and are not restrictive.

1. A method for manufacturing a microstructure, the method comprising the steps of:
(a) forming a water-soluble microstructure on a substrate; and
(b) forming a waterproof thin film on the water-soluble microstructure core.

2. The method of claim 1, wherein the water-soluble microstructure core comprises a drug or an additive.

3. The method of claim 1, wherein the waterproof thin film comprises one or more waterproof polymers selected from the group consisting of a parylene-based polymer, an ethylene-based polymer, an ester-based polymer, an acrylic polymer, an acetyl-based polymer, a styrene-based polymer, a Teflon-based polymer, a vinyl chloride-based polymer, a urethane-based polymer, a nylon-based polymer, a sulfone-based polymer, an epoxy-based polymer, a fluorine-based polymer, and a silicone-based polymer.

4. The method of claim 1, wherein the waterproof thin film has a thickness of 10 nm to 10 μm.

5. The method of claim 1, further comprising the step of (c) depositing a metal onto the waterproof thin film, and then plating the waterproof thin film.

6. The method of claim 5, further comprising the step of (d) separating the substrate, and then removing the water-soluble microstructure core.

7. A microstructure comprising:
a substrate;
a water-soluble microstructure core formed on the substrate; and
a waterproof thin film formed on the water-soluble microstructure core.

8. The microstructure of claim 1, wherein the water-soluble microstructure core comprises a drug or an additive.

9. The microstructure of claim 1, wherein the waterproof thin film comprises one or more waterproof polymers selected from the group consisting of a parylene-based polymer, an ethylene-based polymer, an ester-based polymer, an acrylic polymer, an acetyl-based polymer, a styrene-based polymer, a Teflon-based polymer, a vinyl chloride-based polymer, a urethane-based polymer, a nylon-based polymer, a sulfone-based polymer, an epoxy-based polymer, a fluorine-based polymer, and a silicone-based polymer.

10. The microstructure of claim 1, wherein the waterproof thin film has a thickness of 10 nm to 10 μm.

11. The microstructure of claim 1, further comprising a metal thin film plated on the waterproof thin film.

12. A microstructure comprising: a hollow-type waterproof thin film; and a metal thin film plated on the waterproof thin film.

13. The microstructure of claim 12, wherein the waterproof thin film comprises one or more waterproof polymers selected from the group consisting of a parylene-based polymer, an ethylene-based polymer, an ester-based polymer, an acrylic polymer, an acetyl-based polymer, a styrene-based polymer, a Teflon-based polymer, a vinyl chloride-based polymer, a urethane-based polymer, a nylon-based polymer, a sulfone-based polymer, an epoxy-based polymer, a fluorine-based polymer, and a silicone-based polymer.

14. The microstructure of claim 12, wherein the waterproof thin film has a thickness of 10 nm to 10 μm.
